SEQUENCE LISTING (2 sequences)

SEQ ID NO: 1 - DNA, 1628 bp, Homo sapiens
  CDS (28)..(945)
tttttcctcctcttcctctatactaacatgagtgtggatccagcttgtccccaaagcttgccttgctttgaagaatccgactgtaaagaatcttcacctatgcctgtgatttgtgggcctgaagaaaactatccatccttgcaaatgtcttctgctgagatgcctcacacagagactgtctctcctcttccttcctccatggatctgcttattcaggacagccctgattcttccaccagtcccaaaggcaaacaacccacttctgcagagaatagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcacttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcctatccagtcaatctcatggagggtggagtatggttggagcctaatcagcgaggtttctttttttttttttcctattggatcttcctggagaaaatacttttttttttttttttgagacggagtcttgctctgtcgcccaggctggagtgcagtggcgcggtcttggctcactgcaagctccgcctcccgggttcacgccattctcctgcctcagcctcccgagcagctgggactacaggcgcccgccacctcgcccggctaatattttgtatttttagtagagacagggtttcactgtgttagccaggatggtctcgatctcctgaccttgtgatccgcccgcctcggcctccctaacagctgggattacaggcgtgagccaccgcgccctgcctagaaaagacattttaataaccttggctgctaaggacaacattgatagaagccgtctctggctatagataagtagatctaatactagtttggatatctttagggtttagaatctaacctcaagaataagaaatacaagtacgaattggtgatgaagatgtatt

SEQ ID NO: 2 - protein, 305 aa, Homo sapiens
MSVDPACPQSLPCFEESDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMHFQPNSPASDLEAALEAAGEGLNVIQQTTRYFSTPQTMDLFLNYSMNMQPEDV

(19) United States
(12) Patent Application Publication
(10) Pub. No.: US 2012/0156226 A1
(43) Pub. Date: Jun. 21, 2012

(54) Marker Differentially Expressed in Cancer Stem Cells and Methods of Using Same

(76) Inventor: Kiminobu Sugaya, Winter Park, FL (US)

(21) Appl. No.: 13/321,097

(22) PCT Filed: May 21, 2010
(86) PCT No.: PCT/US2010/035800

§ 371 Date: Mar. 1, 2012

Related U.S. Application Data
(60) Provisional application No. 61/180,276, filed on May 21, 2009.

(51) Int. Cl. A61K 39/395; A61K 31/713; G01N 33/574; C07H 21/04; C12Q 1/18; A61K 38/02; A61P 35/02; C07H 21/02; C12Q 1/04; A61P 35/00; C07K 16/18

(52) U.S. Cl. 424/174.1; 424/178.1; 514/44 A; 436/501; 536/24.5; 530/389.7; 514/19.3; 435/34; 435/32

(57) ABSTRACT

The present invention pertains to methods and agents that target nanog expression or activity for treating or preventing cancer. Alternative methods involve diagnosing cancer stage or type by identifying presence of cancer cells expressing nanog. Other embodiments relate to methods of identifying agents that modulate nanog.

### RELATED APPLICATIONS

This application claims priority to U.S. Ser. No. 61/180,276, filed May 21, 2009, which is incorporated herein in its entirety.

### BACKGROUND

Cancer is one of the most significant health conditions. The American Cancer Society's Cancer Facts and Figures, 2003, predicts over 1.3 million Americans will receive a cancer diagnosis this year. In the United States, cancer is second only to heart disease in mortality accounting for one of four deaths. In 2002, the National Institutes of Health estimated total costs of cancer totaled $171.6 billion, with $61 billion in direct expenditures. The incidence of cancer is widely expected to increase as the US population ages, further augmenting the impact of this condition. The current treatment regimens for cancer, established in the 1970s and 1980s, have not changed dramatically. These treatments, which include chemotherapy, radiation and other modalities including newer targeted therapies, have shown limited overall survival benefit when utilized in most advanced stage common cancers since, among other things, these therapies primarily target tumor bulk rather than cancer stem cells.

More specifically, conventional cancer diagnosis and therapies to date have attempted to selectively detect and eradicate neoplastic cells that are largely fast-growing (i.e., cells that form the tumor bulk). Standard oncology regimens have often been largely designed to administer the highest dose of irradiation or a chemotherapeutic agent without undue toxicity, i.e., often referred to as the “maximum tolerated dose” (MTD) or “no observed adverse effect level” (NOAEL). Many conventional cancer chemotherapies (e.g., alkylating agents such as cyclophosphamide, antimetabolites such as 5-Fluorouracil, plant alkaloids such as vincristine) and conventional irradiation therapies exert their toxic effects on cancer cells largely by interfering with cellular mechanisms involved in cell growth and DNA replication. Chemotherapy protocols also often involve administration of a combination of chemotherapeutic agents in an attempt to increase the efficacy of treatment. Despite the availability of a large variety of chemotherapeutic agents, these therapies have many drawbacks (see, e.g., Stockdale, 1998, “Principles Of Cancer Patient Management” in Scientific American Medicine, vol. 3, Rubenstein and Federman, eds., ch. 12, sect. X). For example, chemotherapeutic agents are notoriously toxic due to non-specific side effects on fast-growing cells whether normal or malignant; e.g. chemotherapeutic agents cause significant, and often dangerous, side effects, including bone marrow depression, immunosuppression, gastrointestinal distress, etc.

Other types of traditional cancer therapies include surgery, hormonal therapy, immunotherapy, epigenetic therapy, anti-angiogenesis therapy, targeted therapy (e.g. therapy directed to a cancer target such as Gleevec®and other tyrosine kinase inhibitors, Velcade®, Sutent®, et al.), and radiation treatment to eradicate neoplastic cells in a patient (see, e.g., Stockdale, 1998, “Principles of Cancer Patient Management,” in Scientific American: Medicine, vol. 3, Rubenstein and Federman, eds., ch. 12, sect. IV). All of these approaches can pose significant drawbacks for the patient including a lack of efficacy (in terms of long-term outcome (e.g. due to failure to target cancer stem cells) and toxicity (e.g. due to non-specific effects on normal tissues)). Accordingly, new therapies and/or regimens for improving the long-term prospect of cancer patients are needed.

Cancer stem cells comprise a unique subpopulation (often 0.1-10% or so) of a tumor that, relative to the remaining 90% or so of the tumor (i.e., the tumor bulk), are more tumorigenic, relatively more slow-growing or quiescent, and often relatively more chemoresistant than the tumor bulk. Given that conventional therapies and regimens have, in large part, been designed to attack rapidly proliferating cells (i.e. those cancer cells that comprise the tumor bulk), cancer stem cells which are often slow-growing may be relatively more resistant than faster growing tumor bulk to conventional therapies and regimens. Cancer stem cells can express other features which make them relatively chemoresistant such as multi-drug resistance and anti-apoptotic pathways. The aforementioned would constitute a key reason for the failure of standard oncology treatment regimens to ensure long-term benefit in most patients with advanced stage cancers—i.e. the failure to adequately target and eradicate cancer stem cells. In some instances, a cancer stem cell(s) is the founder cell of a tumor (i.e., it is the progenitor of the cancer cells that comprise the tumor bulk).

### DETAILED DESCRIPTION

In one aspect, the invention provides a method of treating cancer in a patient in need thereof, the method comprising administering a therapeutically effective regimen, the regimen comprising administering to the patient an antibody that binds to nanog, wherein the patient has been diagnosed with cancer. A non-limiting list of cancers to be treated include urothelial carcinoma, cervical cancer, hematologic cancers, such as leukemia and myeloma, thyroid carcinoma, adenoid cystic carcinoma, breast carcinoma, ovarian cancer, prostate cancer, colon cancer, pancreatic cancer, lymphoma, and neuroblastoma leukemia.

In some embodiments, the patient receives a conventional therapy for the treatment of the cancer before, during or after the administration of the therapeutically effective regimen of the invention, the regimen comprising administering to the patient an agent that modulates the expression or activity of nanog, either directly or indirectly (referred to herein as a “nanog modulating agent”). A non-limiting list of categories of nanog modulating agents includes, siRNA or ribozymes that disrupt expression of nanog, or transcription factors that modulate expression of nanog, or agents that bind directly to nanog that affect its activity. A non-limiting list of examples of such a conventional therapy include chemotherapy, radioimmunotherapy, hormonal therapy, small molecule therapy, toxin therapy, prodrug-activating enzyme therapy, biologic therapy, antibody therapy, surgical therapy, including immunotherapy, anti-angiogenic therapy, targeted therapy, epigenetic therapy, demethylation therapy, histone deacetylase inhibitor therapy, differentiation therapy, radiation therapy, and/or any combination thereof

In another aspect, the invention provides a method of treating cancer in a patient, the method comprising administering to a patient in need thereof a nanog modulating agent, wherein the patient is in remission for the cancer. In yet other aspects, the patient has been previously treated with conventional chemotherapeutic agents or had radiation therapy. In yet another aspect, the patient can be treated with the regimens of the invention following, during or prior to the administration of a conventional chemotherapeutic agent or radiation therapy. In yet another aspect, the patient, concurrent with treatment with the regimens of the invention, can be administered a conventional chemotherapeutic agent or can undergo radiation therapy. Further, the cancer can be refractory or multi-drug resistant. In other aspects, the patient can be treated locally with the methods of the invention. For example, a bladder cancer patient could be treated with the invention via local delivery directly into the tumor, or into the bladder. Local treatment with the invention may also be administered in combination, before, or after other local treatments as well (e.g. BCG therapy).

In yet another aspect, the invention provides a method for preventing a recurrence of cancer in a patient in remission, the method comprising administering to a patient in need thereof a prophylactically effective regimen, the regimen comprising administering to the patient a nanog modulating agent. In another aspect, the invention provides a method for preventing a recurrence of cancer in a patient that has already undergone conventional cancer treatment, the method comprising administering to a patient in need thereof a prophylactically effective regimen, the regimen comprising administering to the patient a nanog modulating agent.

In another embodiment, the invention provides a method for preventing cancer in a patient that is at a high risk for developing cancer, i.e., a patient that has been diagnosed with a nanog-positive precancerous lesion, the method comprising administering to a patient in need thereof a prophylactically effective regimen, the regimen comprising administering to the patient a nanog modulating agent.

In a specific aspect, the methods of the invention can further comprise monitoring the amount of cancer cells or cancer stem cells expressing nanog in a patient undergoing cancer treatment. The methods of the invention may further comprise determining a course of treatment based on the amount of cancer cells or cancer stem cells expressing nanog detected in the patient. The cancer or cancer stem cells may be detected in the patient or in a specimen obtained from the patient. In some embodiments, the specimen is a blood specimen, bone marrow sample, a tissue biopsy, or a tumor biopsy. The amount of cancer cells or cancer stem cells present in the patient or in a sample obtained from the patient can be compared to those present in a reference sample or a sample of cancer cells or cancer stem cells obtained from the patient before or during cancer treatment. In a specific embodiment, the amount of cancer cells or cancer stem cells expressing nanog is monitored using an antibody that binds to nanog.

In another aspect, the invention provides a method of treating a solid tumor in a patient, the method comprising administering to a patient in need thereof a therapeutically effective regimen, the regimen comprising administering to the patient an antibody that binds to the nanog wherein the patient has been diagnosed with a solid tumor, and wherein the patient has undergone primary therapy to reduce the bulk of the tumor. In some embodiments, the primary therapy is, for example, chemotherapy, radioimmunotherapy, hormonal therapy, small molecule therapy, biologic therapy, toxin therapy, prodrug-activating enzyme therapy, antibody therapy, surgical therapy, immunotherapy, anti-angiogenic therapy, targeted therapy, differentiation therapy, epigenetic therapy, demethylation therapy, histone deacetylase inhibitor therapy, radiation therapy, or any combination thereof

In particular embodiments of this aspect, the solid tumor is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiform, astrocytoma, medulloblastoma, craniopharyngioma, ependymoma, pinealoma, hemangioblastoma, acoustic neuroma, oligodendroglioma, meningioma, skin cancer, melanoma, neuroblastoma, or retinoblastoma.

The present invention also provides antibody conjugates comprising an antibody that binds to nanog linked to a therapeutic agent, a cytotoxic agent or other moiety, and compositions comprising such conjugates and uses of such conjugates, including the treatment of a cancer associated with nanog-expressing cells. In some embodiments, an antibody conjugate of the invention comprises an agent that is non-proteinaceous, such as a chemotherapeutic agent or radionuclide. In accordance with these embodiments, the agent can be chemically conjugated to the antibody, either directly or through a chemical linker. In other embodiments, an antibody conjugate of the invention comprises an agent that is proteinaceous. In accordance with these embodiments, the cytotoxic agent can be covalently linked to the antibody through either a peptide bond or other chemical conjugation. The antibody conjugate can be a recombinantly expressed protein that is generated by the linking via molecular biology techniques of the genes for the antibody (or antibody fragment) with the protein toxin, such that the antibody-conjugate is expressed as a single polypeptide chain containing two domains. Non-limiting examples of agents include diphtheria toxin, Pseudomonas exotoxin, ribosome inactivating proteins, Rnase, ricin A, deglycosylated ricin A chain, abrin, alpha sarcin, aspergillin, restrictocin, ribonucleases, bacterial endotoxin, the lipid A moiety of bacterial endotoxin, bouganin, and cholera toxin. Other examples of cytotoxic agents include, but are not limited to, peptides derived from proteins involved in apoptosis, such as Bcl-x, Bax, or Bad. In one embodiment, the cytotoxic agent is Pseudomonas exotoxin A or a fragment thereof In a specific embodiment, the cytotoxic agent is a fragment of Pseudomonas exotoxin A that lacks the native receptor binding domain and contains the translocation and ADP-ribosylation domains of Pseudomonas exotoxin A. In another specific embodiment, the cytotoxic agent is a fragment of Pseudomonas exotoxin A that has been modified at its carboxyl terminus so that it has the amino acid sequence Lys-Asp-Glu-Leu (KDEL).

In some embodiments of the above-described aspects, the regimens comprise the administration of a nanog modulating agent over a period of 1 to 2 weeks, 1 to 3 months, 3 to 6 months, 1 to 12 months, or 6 to 12 months. In some other embodiments the regimens comprise the administration of a nanog modulating agent over a longer period of time such as 9, 12, 24, 36, or 48 months or for the remainder of the patient's life.

As used herein, the term “agent” refers to any molecule, compound, and/or substance for use in the treatment and/or diagnosis of cancer.

As used herein, the terms “about” or “approximately”, unless otherwise indicated, refer to a value that is no more than 10% above or below the value being modified by the term.

As used herein, the term “antibodies” refer to molecules that contain an antigen binding site, e.g., immunoglobulins Immunoglobulin molecules can be of any type (e.g., IgG, IgE, IgM, IgD, IgA and IgY), class (e.g., IgG1, IgG2, IgG3, IgG4, IgA1 and IgA2) or subclass. Antibodies include, but are not limited to, monoclonal antibodies, multispecific antibodies, human antibodies, humanized antibodies, camelized antibodies, chimeric antibodies, single domain antibodies, single chain Fvs (scFv), single chain antibodies, Fab fragments, F(ab′) fragments, disulfide-linked Fvs (sdFv), and anti-idiotopic (anti-Id) antibodies (including, e.g., anti-Id antibodies to antibodies of the invention), and epitope-binding fragments of any of the above. The term antibody will include any protein sequence that confers specificity or binding to its target epitope. Any use of the term antibody will include these permutations. Specific examples of antibodies known to bind to nanog include those available from Santa Cruz biotechnology, Inc. (catalogue nos. sc-33759, sc-81961, sc-30329, sc-33760, sc-30331, sc-30332, or sc-30328)

As used herein, the terms “antibody conjugate(s)” and “antibody fragment conjugate(s)” refer to a conjugate(s) of an antibody or antibody fragment that is prepared by way of a synthetic chemical reaction(s) or as a recombinant fusion protein(s). The term antibody conjugate includes any domain or sequence from an antibody that confers specificity for binding its target, including, but not limited to the permutations described in the definition for “antibody” above.

As used herein, the term “bind” or “bind(s)” refers to any interaction, whether direct or indirect, that affects the specified receptor or receptor subunit.

As used herein, the term “cancer” refers to a neoplasm or tumor resulting from abnormal uncontrolled growth of cells. The term “cancer” encompasses a disease involving both pre-malignant and malignant cancer cells. In some embodiments, cancer refers to a localized overgrowth of cells that has not spread to other parts of a subject, i.e., a benign tumor. In other embodiments, cancer refers to a malignant tumor, which has invaded and destroyed neighbouring body structures and spread to distant sites. In yet other embodiments, the cancer is associated with a specific cancer antigen.

As used herein, the term “cancer cells” refers to cells that acquire a characteristic set of functional capabilities during their development, including the ability to evade apoptosis, self-sufficiency in growth signals, insensitivity to anti-growth signals, tissue invasion/metastasis, significant growth potential, and/or sustained angiogenesis. The term “cancer cell” is meant to encompass both pre-malignant and malignant cancer cells.

As used herein, the term “cancer stem cell(s)” refers to a cell that can be a progenitor of a highly proliferative cancer cell. A cancer stem cell has the ability to re-grow a tumor as demonstrated by its ability to form tumors in immunocompromised mice, and typically to form tumors upon subsequent serial transplantation in immunocompromised mice. Cancer stem cells are also typically slow-growing relative to the bulk of a tumor; that is, cancer stem cells are generally quiescent. In certain embodiments, but not all, the cancer stem cell may represent approximately 0.1 to 10% of a tumor.

As used herein, the term “compound” refers to any agent that is being tested for its ability to bind to nanog or has been identified as binding to nanog, including the particular antibodies provided herein or incorporated by reference herein. In one embodiment, a compound is purified (e.g., 85%, 90%, 95%, 99%, or 99.9% pure). Such compounds for example, generally include any agent comprised of two or more atoms or ions of two or more elements in chemical combination wherein the constituents are united by bonds or valence forces (see Hawley's Condensed Chemical Dictionary, Thirteenth Edition, 1997). Non-limiting examples of compounds include, but are not limited to, proteinaceous molecules, including, but not limited to, peptides (including dimers and multimers of such peptides), polypeptides, proteins, including post-translationally modified proteins, conjugates, antibodies, antibody fragments, antibody conjugates, small molecules, including inorganic or organic compounds; nucleic acid molecules including, but not limited to, double-stranded or single-stranded DNA, or double-stranded or single-stranded RNA, antisense RNA, RNA interference (RNAi) molecules (e.g., small interfering RNA (siRNA), micro-RNA (miRNA), short hairpin RNA (shRNA), etc.), intron sequences, triple helix nucleic acid molecules and aptamers; carbohydrates; and lipids.

As used herein, the term “cytotoxin” or the phrase “cytotoxic agent” refers to an antibody that exhibits an adverse effect on cell growth or viability. Included in this definition are compounds that kill cells or which impair them with respect to growth, longevity, or proliferative activity.

As used herein, the term “derivative” in the context of proteinaceous agent (e.g., proteins, polypeptides, peptides, and antibodies) refers to a proteinaceous agent that comprises an amino acid sequence which has been altered by the introduction of amino acid residue substitutions, deletions, and/or additions. The term “derivative” as used herein also refers to a proteinaceous agent which has been modified, i.e., by the covalent attachment of any type of molecule to the proteinaceous agent. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may be produced by chemical modifications using techniques known to those of skill in the art, including, but not limited to specific chemical cleavage, acetylation, formylation, metabolic synthesis in the presence of tunicamycin, etc. Further, a derivative of a proteinaceous agent may contain one or more non-classical amino acids. A derivative of a proteinaceous agent possesses a similar or identical function as the proteinaceous agent from which it was derived. The term “derivative” in the context of a proteinaceous agent also refers to a proteinaceous agent that possesses a similar or identical function as a second proteinaceous agent (i.e., the proteinaceous agent from which the derivative was derived) but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a proteinaceous agent can be determined by methods known to those skilled in the art, including but not limited to, peptide sequencing, X-ray crystallography, nuclear magnetic resonance, circular dichroism, and crystallographic electron microscopy. In a specific embodiment, a derivative is a functionally active derivative.

As used herein, the phrase “diagnostic agent” refers to any molecule, compound, and/or substance that is used for the purpose of diagnosing cancer. Non-limiting examples of diagnostic agents include antibodies, antibody fragments, or other proteins, including those conjugated to a detectable agent. As used herein, the term “detectable agents” refer to any molecule, compound and/or substance that is detectable by any methodology available to one of skill in the art. Non-limiting examples of detectable agents include dyes, gases, metals, or radioisotopes.

As used herein, the term “effective amount” refers to the amount of a therapy that is sufficient to result in the prevention of the development, recurrence, or onset of cancer and one or more symptoms thereof, to enhance or improve the prophylactic effect(s) of another therapy, reduce the severity, the duration of cancer, ameliorate one or more symptoms of cancer, prevent the advancement of cancer, cause regression of cancer, and/or enhance or improve the therapeutic effect(s) of another therapy. In an embodiment of the invention, the amount of a therapy is effective to achieve one, two, three or more of the following results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate; (10) a decrease in hospitalization lengths; (11) the size of the tumor is maintained and does not increase or increases by less than 10%, preferably less than 5%, preferably less than 4%, preferably less than 2%; (12) an increase in the number of patients in remission; (13) an increase in the length or duration of remission; (14) a decrease in the recurrence rate of cancer; (15) an increase in the time to recurrence of cancer; and (16) an amelioration of cancer-related symptoms and/or quality of life.

As used herein, the terms “subject” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the term “therapeutic agent” refers to any molecule, compound, and/or substance that is used for the purpose of treating and/or managing cancer. Examples of therapeutic agents include, but are not limited to, proteins, immunoglobulins (e.g., multi-specific Igs, single chain Igs, Ig fragments, polyclonal antibodies and their fragments, monoclonal antibodies and their fragments), antibody conjugates or antibody fragment conjugates, peptides (e.g., peptide receptors, selectins), binding proteins, chemospecific agents, chemotoxic agents (e.g., anti-cancer agents), radiation, chemotherapy, anti-angiogenic agents, and small molecule drugs. Therapeutic agents may be a(n) anti-angiogenesis therapy, targeted therapy, radioimmunotherapy, small molecule therapy, biologic therapy, epigenetic therapy, toxin therapy, differentiation therapy, pro-drug activating enzyme therapy, antibody therapy, chemotherapy, radiation therapy, hormonal therapy, immunotherapy, or protein therapy.

As used herein, the terms “therapies” and “therapy” can refer to any method(s), composition(s), and/or agent(s) that can be used in the treatment of a cancer or one or more symptoms thereof. In certain embodiments, the terms “therapy” and “therapies” refer to chemotherapy, radiation therapy, radioimmunotherapy, hormonal therapy, targeted therapy, toxin therapy, pro-drug activating enayme therapy, protein therapy, antibody therapy, small molecule therapy, epigenetic therapy, demethylation therapy, histone deacetylase inhibitor therapy, differentiation therapy, antiangiogenic therapy, biological therapy including immunotherapy and/or other therapies useful in the treatment of a cancer or one or more symptoms thereof

As used herein, the terms “treat”, “treatment”, and “treating” in the context of the administration of a therapy to a subject refer to the reduction or inhibition of the progression and/or duration of cancer, the reduction or amelioration of the severity of cancer, and/or the amelioration of one or more symptoms thereof resulting from the administration of one or more therapies. In a specific embodiment, a patient that is at a high risk for developing cancer is treated, i.e., a patient that has been diagnosed with a nanog positive precancerous lesion. In specific embodiments, such terms refer to one, two, or three or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate; (10) a decrease in hospitalization lengths; (11) the size of the tumor is maintained and does not increase or increases by less than 10%, preferably less than 5%, preferably less than 4%, preferably less than 2%; (12) an increase in the number of patients in remission; (13) an increase in the length or duration of remission; (14) a decrease in the recurrence rate of cancer; (15) an increase in the time to recurrence of cancer; and (16) an amelioration of cancer-related symptoms and/or quality of life. In certain embodiments, such terms refer to a stabilization or reduction in the cancer stem cell population. In some embodiments, such terms refer to a stabilization or reduction in the growth of cancer cells. In some embodiments, such terms refer to a stabilization or reduction in the cancer stem cell population and a reduction in the cancer cell population. In some embodiments, such terms refer to a stabilization or reduction in the growth and/or formation of a tumor. In some embodiments, such terms refer to the eradication, removal, or control of primary, regional, or metastatic cancer (e.g., the minimization or delay of the spread of cancer). In some embodiments, such terms refer to a reduction in mortality and/or an increase in survival rate of a patient population. In further embodiments, such terms refer to an increase in the response rate, the durability of response, or number of patients who respond or are in remission. In some embodiments, such terms refer to a decrease in hospitalization rate of a patient population and/or a decrease in hospitalization length for a patient population.

The present invention provides antibody conjugates that bind to nanog. In some embodiments, the antibody conjugates of the present invention comprise an antibody that binds to nanog conjugated to a therapeutic agent, a cytotoxic agent or other moiety (e.g., an anticellular moiety). In some embodiments, the antibody conjugates of the present invention comprise an antibody that binds to nanog conjugated to a therapeutic agent, a cytotoxic agent or other moiety (e.g., an anticellular moiety). In one embodiment, the antibody is conjugated to a cytotoxic agent or otherwise anticellular agent, either directly or through a chemical linker. In another embodiment, the antibody is linked to the cytotoxic agent or otherwise anticellular or anticancer moiety through a chemical (covalent) bond, a recombinant antibody conjugate, such as a peptide bond (with or without a peptide linker), disulfide bond, or sterically hindered disulfide bond. The antibody can be linked at its amino terminus or its carboxyl terminus to the cytotoxic agent or otherwise anticellular or anticancer moiety. Alternatively, the antibody can replace a domain of the cytotoxic agent or otherwise anticellular moiety that is not required for cytotoxicity so long as the antibody retains its specificity for nanog.

Any cytotoxic agent or otherwise anticellular agent known to one of skill in the art can be used to produce the antibody conjugates of the invention. A cytotoxic agent includes any agent that is detrimental to cells. Exemplary cytotoxic agents include chemotherapeutic agents, radioisotopes, cytotoxins such as cytostatic or cytocidal agents, or other anticellular agents, including known therapeutic agents.

Non-limiting examples of cytotoxic agents include antimetabolites (e.g., cytosine arabinoside, aminopterin, methotrexate, 6-mercaptopurine, 6-thioguanine, cytarabine, and 5-fluorouracil decarbazine); alkylating agents (e.g., mechlorethamine, thiotepa chlorambucil, melphalan, carmustine (BCNU) and lomustine (CCNU), cyclophosphamide, busulfan, dibromomannitol, streptozotocin, mitomycin C, cis-dichlorodiammine-platinum (II) (CDDP), and cisplatin); vinca alkaloid; anthracyclines (e.g., daunorubicin (formerly daunomycin) and doxorubicin); antibiotics (e.g., dactinomycin (formerly actinomycin), bleomycin, mithramycin, and anthramycin (AMC)); calicheamicin; CC-1065 and derivatives thereof; auristatin molecules (e.g., auristatin PHE, bryostatin-1, and dolastatin-10; see Woyke, et al., Antimicrob Agents Chemother 46:3802-8 (2002), Woyke, et al., Antimicrob Agents Chemother 45:3580-4 (2001), Mohammad, et al., Anticancer Drugs 12:735-40 (2001), Wall, et al., Biochem Biophys Res Commun 266:76-80 (1999), Mohammad, et al., Int J Oncol 15:367-72 (1999), all of which are incorporated herein by reference); DNA-repair enzyme inhibitors (e.g., etoposide or topotecan); kinase inhibitors (e.g., compound ST1571, imatinib mesylate (Kantarjian, et al., Clin Cancer Res 8(7):2167-76 (2002)); demecolcine; and other cytotoxic agents (e.g., paclitaxel, cytochalasin B, gramicidin D, ethidium bromide, emetine, mitomycin, etoposide, tenoposide, vincristine, vinblastine, colchicine, doxorubicin, daunorubicin, dihydroxy anthracenedione, mitoxantrone, mithramycin, actinomycin D, 1-dehydrotestosterone, glucorticoids, procaine, tetracaine, lidocaine, propranolol, and puromycin and analogs or homologues thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459); farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305); topoisomerase inhibitors (e.g., camptothecin, irinotecan, SN-38, topotecan, 9-aminocamptothecin, GG211 (GI147211), DX-8951f, IST-622, rubitecan, pyrazoloacridine, XR5000, saintopin, UCE6, UCE1022, TAN-1518A, TAN 1518B, KT6006, KT6528, ED-110, NB-506, ED-110, NB-506, and rebeccamycin); bulgarein; DNA minor groove binders such as Hoechst dye 33342 and Hoechst dye 33258; nitidine; fagaronine; epiberberine; coralyne; beta-lapachone; BC-4-1; antisense oligonucleotides (e.g., those disclosed in the U.S. Pat. Nos. 6,277,832, 5,998,596, 5,885,834, 5,734,033, and 5,618,709); adenosine deaminase inhibitors (e.g., fludarabine phosphate and 2-chlorodeoxyadenosine); and pharmaceutically acceptable salts, solvates, clathrates, and prodrugs thereof.

The compositions of the invention can be in the form of a solid, liquid or gas (aerosol). Typical routes of administration may include, without limitation, oral, topical, parenteral, sublingual, rectal, vaginal, ocular, intradermal, intratumoral, intracerebral, intrathecal, and intranasal. Parenteral administration includes subcutaneous injections, intravenous, intramuscular, intraperitoneal, intrapleural, intrasternal injection, directly into the lumen of the bladder, directly into the tumor, or infusion techniques. In a specific embodiment, the compositions are administered parenterally. In a more specific embodiment, the compositions are administered intravenously. Pharmaceutical compositions of the invention can be formulated so as to allow an antibody of the invention to be bioavailable upon administration of the composition to a subject. Compositions can take the form of one or more dosage units, where, for example, a tablet can be a single dosage unit, and a container of an antibody of the invention in aerosol form can hold a plurality of dosage units.

In one embodiment, the nanog antibody is conjugated to a radioactive metal ion, such as the alpha-emitters ²¹¹astatine, ²¹²bismuth, ²¹³bismuth; the beta-emitters ¹³¹iodine, ⁹⁰yttrium, ¹⁷⁷lutetium, ¹⁵³samarium, and ¹⁰⁹palladium; or macrocyclic chelators useful for conjugating radiometal ions, including but not limited to, ¹³¹indium ¹³¹L, ¹³¹yttrium, ¹³¹holmium, ¹³¹samarium, to polypeptides or any of those listed supra. In certain embodiments, the macrocyclic chelator is 1,4,7,10-tetraazacyclododecane-N,N′,N″,N′″-tetraacetic acid (DOTA), which can be attached to the antibody via a linker molecule. Such linker molecules are commonly known in the art and described in Denardo, et al., 1998, Clin Cancer Res 4 (10):2483-90; Peterson, et al., 1999, Bioconjug Chem 10 (4):553-7; and Zimmerman, et al., 1999, Nucl Med Biol 26 (8):943-50, each incorporated by reference in their entireties.

### Isolation

Certain embodiments of the present inventions are based on the inventors discovery that nanog is differentially expressed in cancer stem cells. Based on this, isolation of cancer stem cells may be accomplished by positive selection, negative selection or through histological/growth characteristics. In one embodiment, nanog is the marker used for positive selection of cells, such as through flow cytometry or magnetic separation. Conversely, markers absent in cancer stem cells, but present in other cells in tumor, may be used to negatively select out cells other than cancer stem cells.

Antibodies can be produced that bind to nanog. Once at least one successful antibody per group is determined, those antibodies are used to select out subpopulations of cells from tumor samples. This may be accomplished by attaching magnetic particles to antibodies and incubating the conjugated antibodies with cells isolated from the tumor. Following incubation, the cells are run through a magnetic column to separate out cells attached to a magnetic antibody (because of expression of a target surface protein) and non-attached cells will flow through the column. This technique enables purification of individual cell populations within the tumor for further study.

Furthermore, different cells in a tumor sample may be isolated based on their histological or growth characteristics. For example, cells from a tumor sample may be adherent to surfaces compared to other cells. Adherent cells are in most cases more differentiated tumor cells not cancer stem cells. Cancer stem cells also may have a propensity to form spheres. Cells tending to form spheres can be selected apart from cells not tending to form spheres. Cells may also be isolated based on the hanging-drop method. Tissue Engineering, Second Edition, Hauser and Fussenegger, 2007, Human Press.

### Immunotherapy

According to another embodiment, the invention pertains to a method of conducting immunotherapy involving the administration of activated antigen presenting cells. In another embodiment, the invention involves the creation of antigen presenting cells (APCs) activated against cancer stem cells. As used herein, antigen presenting cells include but are not limited to dendritic cells, macrophages or natural killer cells. Other examples of cells that could serve as antigen presenting cells, include fibroblasts, glial cells and microglial cells.

In one example, dendritic cells are activated against markers and antigens present in cancer stem cells. APCs are contacted with the marker or antigen, such as nanog, they are taken into the cell, processed and then presented on the surface of the cell. In another example, mRNA or DNA in CSCs is subjected to APCs, which also results in an activation against the CSCs from which the mRNA and/or DNA was procured. In another example, dendritic cells are activated by fusion with a CSC. The antigen presenting cells take in and digest the cancer stem cells by phagocytosis and/or endocytosis. Alternatively, or in conjunction with phagocytosis and/or endocytosis, the dendritic cells are subjected to electrical current in the presence of the CSCs.

In another embodiment, a tumor sample containing multiple cell types is procured from a subject. As has been discussed herein, it is the inventors' belief that if cancer stem cells can be preferentially targeted over other cells in a tumor this will dramatically improve cancer therapy. Accordingly, cancer stem cells are isolated or enriched from the tumor sample. Tumor samples may be procured from an allogeneic source, i.e., a subject of the same species but other than the subject into which activated antigen presenting cells are administered. In other embodiments, the tumor samples are procured from an autologous source. For example, tumor cells are removed from a cancer subject, the cells are used to activate antigen presenting cells ex vivo and then the activated cells are administered to the cancer subject.

Cancer Stem Cell lines

In a further embodiment, the subject invention pertains to a plurality of cancer stem cell lines and a facility for storage of such lines. This embodiment is based on the inventors' realization that there is a need for a convenient systematic access to different cancer stem cell lines. The inventors have realized that the ability to identify cancer stem cell lines derived from various tumor types will be exceedingly useful for identifying specific markers for distinguishing cancer stem cells from other cells in a given cancer type. Different cancer stem cell lines will be useful for testing various compounds for their effect on the growth and/or survival of the specific cancer stem cell type. This in turn, will lead to the discovery of potential new cancer therapies. Subjects from which cancer stem cells are procured for establishing a given cell line may be human or nonhuman vertebrates.

In a specific embodiment, a population of cancer stem cells that express nanog is used to screen a number of potential drug candidates. See U.S. Patent Publications 20080014206; 20070142288 for screening techniques and protocols.

According to another embodiment, cancer stem cells are harvested, catalogued according to predetermined characteristics, e.g., phenotypic information, morphological characteristics, differentiation profile, blood type, major histocompatibility complex, disease state of donor, or genotypic information (e.g. single nucleated polymorphisms, ‘SNPs’ of a specific nucleic acid sequence associated with a gene, or genomic or mitochondrial DNA), and stored under appropriate conditions (typically by freezing) to keep the cancer stem cells alive and functioning. Other characteristics may include, resistance to chemotherapies, production of membrane channels that confer drug resistance, surface markers and surface receptors. Cataloguing may constitute creating a centralized record of the characteristics obtained for each cell population, such as, but not limited to, an assembled written record or a computer database with information inputted therein. Essentially, this embodiment pertains to the production of a stem cell bank. The cancer stem cell bank facilitates the selection from a plurality of samples of a specific stem cell sample suitable for a researcher's needs. Thus, another embodiment of the subject invention pertains to a cancer stem cell bank comprising a plurality of cancer stem cell samples obtained from separate sources and which are characterized and catalogued according to at least one predetermined characteristic. An additional embodiment pertains to a method of establishing a cancer stem cell bank comprising collecting cancer stem cell samples from multiple sources; cataloguing the samples according to at least one predetermined characteristic and storing the cancer stem cells under conditions that keep cells viable.

The present invention provides methods for stabilizing, reducing or eliminating a cancer stem cell population. In particular, the present invention provides methods for stabilizing, reducing or eliminating a cancer stem cell population in a subject, the method comprising administering to a subject in need thereof a prophylactically or therapeutically effective regimen, the regimen comprising administering one or more therapies to the subject. In certain embodiments, the regimen results in the stabilization of a cancer stem cell population as assessed by methods such as those described in Section 4.3, infra, after a period and/or duration of certain survival endpoints. Thus, in order to achieve stabilization, reduction, or elimination in the growth, size, and/or formation of a tumor and/or metastases by stabilizing, reducing or eliminating the cancer stem cell population, a therapy can be administered for a longer period of time, and in some embodiments, more frequently or more continuously than currently administered or known to one of skill in the art. In certain embodiments, a lower dose than currently used or known to one of skill in the art is administered for a longer period of time, and in some embodiments, more frequently or more continuously than currently administered or known to one of skill in the art.

### Other Therapies

In other embodiments, The present invention provides methods for stabilizing, reducing, or eliminating the cancer stem cells and the cancer cells in a subject, the method comprising administering to a subject in need thereof a prophylactically or therapeutically effective regimen, the regimen comprising administering one or more therapies to the subject. In one embodiment, the regimen achieves a 5%-40%, preferably a 10%-60%, and more preferably a 20 to 99% reduction in the cancer stem cell population, and/or a 5%-40%, preferably a 10%-60%, and more preferably at 20 to 99% reduction in the cancer cell population. In a specific embodiment, the reduction in the cancer stem cell population and/or the cancer cell population is achieved after two weeks, a month, two months, three months, four months, six months, nine months, 1 year, 2 years, 3 years, 4 years, or more of administration of one or more therapies.

The present invention provides methods for stabilizing or reducing the population of cancer stem cells and the bulk size of a tumor in a subject, the methods comprising administering to a subject in need thereof a prophylactically or therapeutically effective regimen, the regimen comprising administering one or more therapies to the subject. In one embodiment, the regimen achieves a 5%-40%, preferably a 10%-60%, and more preferably a 20 to 99% reduction in the cancer stem cell population, and/or a 5%-40%, preferably a 10%-60%, and more preferably a 20 to 99% reduction in the bulk size of the tumor. In a specific embodiment, the reduction in the cancer stem cell population and/or tumor size is achieved after two weeks, a month, two months, three months, four months, six month, nine months, 1 year, 2 years, 3 years, 4 years, or more of administration of one or more of the therapies. In a of time (e.g., after 2, 5, 10, 20, 30 or more doses of a therapy, or after 2 weeks, 1 month, 2 months, 1 year, 2 years, 3 years, 4 years or more). In other embodiments, the regimen achieves a 5%-40%, preferably a 10%-60%, and more preferably a 20 to 99% reduction in the cancer stem cell population. In some embodiments, the reduction in a cancer stem cell population is achieved after two weeks, a month, two months, three months, four months, six month, nine months, 1 year, 2 years, 3 years, or 4 years of administration of one or more therapies. In certain embodiments, in accordance with the regimen, the reduction in a cancer stem cell population is monitored periodically (e.g., after 2, 5, 10, 20, 30 or more doses of one or more therapies, or after 2 weeks, 1 month, 2 months, 1 year, 2 years, 3 years, 4 years or more after receiving one or more therapies).

Without being bound by a particular theory or mechanism, the stabilization, reduction or elimination of a cancer stem cell population stabilizes, reduces or eliminates the cancer cell population produced by the cancer stem cell population, and thus, stabilizes, reduces or eliminates the growth of a tumor, the bulk size of a tumor, the formation of a tumor and/or the formation of metastases. In other words, the stabilization, reduction or elimination of the cancer stem cell population prevents the formation, reformation or growth of a tumor and/or metastases by cancer cells.

Cancer stem cells can proliferate relatively slowly so that conventional therapies and regimens that differentially impair, inhibit or kill rapidly proliferating cell populations (e.g., cancer cells comprising the tumor bulk) in comparison with cell populations that divide more slowly, most likely do not effectively target and impair cancer stem cells. The methods and regimens of the present invention are designed to result in a concentration (e.g., in blood, plasma, serum, tissue, and/or tumor) of a therapy(ies) that will stabilize or reduce a cancer stem cell population.

Since cancer stem cells often make up only a subpopulation of a tumor, a therapy that stabilizes, reduces or eliminates cancer stem cells may require a longer period of time than is traditionally expected for a cancer patient to achieve stabilization, reduction or elimination in the growth, size and/or formation of a tumor and/or metastases, or an amelioration of cancer-related symptoms. Accordingly, during this additional time period, there is an opportunity to deliver additional therapy, albeit at less toxic (e.g., lower) doses. As a result of stabilizing, reducing, or eliminating the cancer stem cell population, the cancer may be significantly impaired, the frequency of responses increased albeit potentially occurring at later time points, the duration of a remission increased, and/or the frequency particular embodiment, the reduction in the cancer stem cell population is determined by a method described, infra, and the bulk size of the tumor is measured by methods known to one of skill in the art. Non-limiting examples of methods for measuring the bulk size of a tumor include radiological methods (e.g., computed tomography (CT), MRI, X-ray, mammogram, PET scan, radionuclide scan, bone scan), visual methods (e.g., colonoscopy, bronchoscopy, endoscopy), physical exam (e.g., prostate, breast, lymph nodes, abdominal, general palpation), blood tests (e.g., PSA, CEA, CA-125, AFP, liver function tests), bone marrow analysis (e.g., in the case of a hematological malignancy), histopathology, cytology, and flow cytometry. In certain embodiments, in accordance with the regimen, the cancer stem cell population and/or the tumor size are monitored periodically (e.g., after 2, 5, 10, 20, 30, or more doses of one or more of the therapies, or after 2 weeks, 1 month, 2 months, 6 months, 1 year, or more of receiving one or more therapies).

In certain embodiments, the prophylactically and/or therapeutically effective regimens do not affect tumor angiogenesis. In other embodiments, the prophylactically and/or therapeutically effective regimens reduce tumor angiogenesis by less than 25%, preferably less than 15%, and more preferably less than 10%. Tumor angiogenesis can be assessed by techniques known to one of skill in the art, including, e.g., assessing microvessel density of a tumor and measuring the circulating endothelial cell population and the circulating endothelial progenitor population in a blood sample.

The present invention provides methods for stabilizing, reducing, or eliminating the population of cancer stem cells in a subject, the methods comprising administering to a subject in need thereof a prophylactically or therapeutically effective regimen, the regimen comprising administering one or more therapies to the subject, wherein the regimen does not result in a reduction or results in a small reduction in the circulating endothelial cell population. In one embodiment, the regimen achieves 5%-40%, preferably a 10%-60%, and more preferably a 20 to 99% reduction in the cancer stem cell population and less than a 25%, preferably less than a 15%, and more preferably less than a 10% reduction in the circulating endothelial cell population. In a specific embodiment, the reduction in the cancer stem cell population is achieved after two weeks, a month, two months, three months, four months, six month, nine months, 1 year, 2 years, 3 years, 4 years or more of administration of one or more of the therapies. The present invention provides methods for stabilizing, reducing, or eliminating the population of cancer stem cells in a subject, the methods comprising administering to a subject in need thereof a prophylactically or therapeutically effective regimen, the regimen comprising administering one or more therapies to the subject, wherein the regimen does not result in a reduction or results in a small reduction in the circulating endothelial progenitor population.

The present invention provides methods for preventing, treating and/or managing cancer, the methods comprising administering to a subject in need thereof a prophylactically or therapeutically effective regimen, the regimen comprising administering one or more therapies to the subject, wherein the regimen results in at least an approximately 2.5%, 5%, 10%, 15%, 20%, 25%, 30%, 40%, 50%, 60%, 70%, 80%, 90%, 95%, 97.5%, or 99% reduction in the cancer stem cell population. In one embodiment, the regimen achieves a 5%-40%, preferably a 10%-60%, and more preferably a 20 to 99% reduction in the cancer stem cell population. In a specific embodiment, the reduction in the cancer stem cell population is determined by a method described herein. In some embodiments, the reduction in the cancer stem cell population is achieved after two weeks, a month, two months, three months, four months, six month, nine months, 1 year, 2 years, 3 years, 4 years or more of administration of one or more of the therapies. In certain embodiments, in accordance with the regimen, the reduction in the cancer stem cell population is monitored after a period of time (e.g., after 2, 5, 10 or more doses of one or more of the therapies or after 2 weeks, 1 month, 2 months, 6 months, 1 year, or more of receiving one or more therapies).

The present invention provides methods for preventing, treating and/or managing cancer, the methods comprising administering to a subject in need thereof a prophylactically or therapeutically effective regimen, the regimen comprising administering one or more therapies to the subject, wherein the regimen stabilizes the cancer stem cell population. In some embodiments, the stabilization of the cancer stem cell population is achieved after two weeks, a month, two months, three months, four months, six month, nine months, 1 year, 2 years, 3 years, 4 years or more of administration of one or more of the therapies. In certain embodiments, in accordance with the regimen, the stabilization of the cancer stem cell population is monitored after a period of time (e.g., after 2, 5, 10 or more doses of one or more of the therapies or after 2 weeks, 1 month, 2 months, 6 months, 1 year, or more of receiving one or more therapies).

The present invention provides methods of preventing, treating and/or managing cancer, the method comprising: (a) administering to a subject in need thereof one or more doses of an effective amount of a therapy; (b) monitoring the cancer stem cell population in the subject prior to, during, and/or after the administration of a certain number of doses and prior to the administration of a subsequent dose; and (c) maintaining at least a 5%-40%, preferably a 10%-60%, and more preferably a 20 to 99% reduction in the cancer stem cell population in the subject by repeating step (a) as necessary. In a specific embodiment, the reduction in the cancer stem cell population is determined by a method described, infra. In some embodiments, the reduction of the cancer stem cell population is achieved after 5 to 30, 10 to 50, 10 to 75, 10 to 100, 10 to 150, or 10 to 300 doses of the therapy.

The present invention provides methods for preventing, treating and/or managing cancer, the methods comprising administering to a subject in need thereof a prophylactically or therapeutically effective regimen, the regimen comprising administering one or more therapies to the subject, wherein the regimen results in the stabilization or reduction in the cancer stem cell population and a reduction in the bulk size of a tumor. In one embodiment, the regimen achieves a 5%-40%, preferably a 10%-60%, and more preferably a 20 to 99% reduction in the cancer stem cell population, and/or a 5%-40%, preferably a 10%-60%, and more preferably a 20 to 99% reduction in the bulk size of the tumor. In a specific embodiment, the reduction the cancer stem cell population and/or tumor size is achieved after two weeks, a month, two months, three months, four months, six month, nine months, 1 year, 2 years, 3 years, 4 years or more of administration of one or more of the cancer therapies. In a particular embodiment, the stabilization or reduction in the cancer stem cell population is determined by the methods described infra, and the bulk size of the tumor is measured by a method described in infra. In certain embodiments, in accordance with the regimen, the cancer stem cell population and/or the reduction in the tumor size is monitored periodically (e.g., after 2, 5, 10, 20, 30 or more doses of one or more of the therapies or after 2 weeks, 1 month, 2 months, 6 months, 1 year, or more of receiving one or more therapies).

The present invention provides methods of preventing, treating and/or managing cancer, the method comprising: (a) administering to a subject in need thereof one or more doses of an effective amount of a therapy; (b) monitoring the cancer stem cell population and the bulk tumor size in or from the subject prior to, during, and/or after the administration of a certain number of doses and prior to the administration of a subsequent dose; and (c) maintaining at least a 5%-40%, preferably a 10%-60%, and more preferably a 20 to 99% reduction in the cancer stem cell population and at least a 5%-40%, preferably a 10%-60%, and more preferably a 20 to 99% reduction in the reduction in the bulk tumor size in the subject by repeating step (a) as necessary. In a specific embodiment, the reduction in the cancer stem cell population is determined by a method described infra, and the reduction in the bulk tumor size is determined by a method known to one of skill in the art, e.g., conventional CT scans, PET scans, bone scans, MRIs or X-ray imaging, among other methods. In some embodiments, the reduction of the cancer stem cell population and the reduction in the bulk tumor size are achieved after 5-30, 10-50, 10-75, 10 to 100, 10 to 150, or 10 to 300 doses of the therapy or after 2 weeks, 1 month, 2 months, 6 months, 1 year, or more of receiving one or more therapies.

The present invention provides methods of preventing, treating and/or managing cancer, the methods comprising administering to a subject in need thereof a prophylactically or therapeutically effective regimen, the regimen comprising administering one or more therapies to the subject, wherein the regimen results does not result in or results in only a small reduction in the circulating endothelial cell population. In certain embodiments, the regimen results in less than a 25%, preferably less than a 15%, and more preferably less than a 10% reduction in the circulating endothelial cell population. In certain embodiments, the circulating endothelial cell population is monitored periodically (e.g., after 2, 5, 10, 20, 30 or more doses of one or more therapies or after 2 weeks, 1 month, 2 months, 6 months, 1 year, or more of receiving one or more therapies).

The present invention provides methods of preventing, treating and/or managing cancer, the methods comprising administering to a subject in need thereof a prophylactically or therapeutically effective regimen, the regimen comprising administering one or more therapies to the subject, wherein the regimen results does not result in or results in only a small reduction in the circulating endothelial progenitor population. In certain embodiments, the regimen results in less than a 25%, preferably less than a 15%, and more preferably less than a 10% reduction in the circulating endothelial progenitor population. In certain embodiments, the circulating endothelial progenitor population is monitored periodically (e.g., after 2, 5, 10, 20, 30 or more doses of one or more therapies or after 2 weeks, 1 month, 2 months, 6 months, 1 year, or more of receiving one or more therapies).

The present invention provides methods of preventing, treating and/or managing cancer, the methods comprising administering to a subject in need thereof a prophylactically or therapeutically effective regimen, the regimen comprising administering one or more therapies to the subject, wherein the regimen results does not result in or results in only a small reduction in the circulating endothelial cell population and the circulating endothelial progenitor population. In certain embodiments, the regimen results in less than a 25%, preferably less than a 15%, and more preferably less than a 10% reduction in the circulating endothelial cell population and the circulating endothelial progenitor population. In certain embodiments, the circulating endothelial cell population and the circulating endothelial progenitor population are monitored periodically (e.g., after 2, 5, 10, 20, 30 or more doses of one or more therapies or after 2 weeks, 1 month, 2 months, 6 months, 1 year, or more of receiving one or more therapies).

The present invention also provides methods of preventing, treating and/or managing cancer, the methods comprising administering to a subject in need thereof a prophylactically or therapeutically effective regimen, the regimen comprising administering one or more therapies to the subject, wherein the regimen results in the stabilization or reduction in the cancer stem cell population and does not result in a reduction or only results in a small reduction of the circulating endothelial cell population and/or the circulating endothelial progenitor population. In one embodiment, the regimen achieves a 5%-40%, preferably a 10%-60%, and more preferably at 20 to 99% reduction in the cancer stem cell population and/or less than a 25%, preferably less than a 15%, and more preferably less than a 10% reduction in the circulating endothelial cell population. In another embodiment, the regimen achieves a 5%-40%, preferably a 10%-60%, and more preferably at 20 to 99% reduction in the cancer stem cell population and/or less than a 25%, preferably less than a 15%, and more preferably less than a 10% reduction in the circulating endothelial progenitor population. In another embodiment, the regimen achieves a 5%-40%, preferably a 10%-60%, and more preferably at 20 to 99% reduction in the cancer stem cell population and/or less than a 25%, preferably less than a 15%, and more preferably less than a 10% reduction in the circulating endothelial cell population and the circulating endothelial progenitor population. In a specific embodiment, the stabilization or reduction in the cancer stem cell population is achieved after two weeks, a month, two months, three months, four months, six month, nine months, 1 year, 2 years, 3 years, 4 years or more of administration of one or more of the therapies. In a particular embodiment, the stabilization or reduction in the cancer stem cell population is determined by a method described in Section 4.3, infra, and a reduction in the circulating endothelial cell population and/or the circulating endothelial progenitor population is determined by a method described in Section 4.5, infra. In certain embodiments, in accordance with the regimen, the circulating cancer stem cell population, the circulating endothelial cell population and/or the circulating endothelial progenitor population is monitored periodically (e.g., after 2, 5, 10, 20, 30 or more doses of one or more of the therapies or after 2 weeks, 1 month, 2 months, 6 months, 1 year, or more of receiving one or more therapies).

The present invention provides methods for preventing, treating and/or managing cancer, the methods comprising administering a prophylactically and/or therapeutically effective regimen to a subject in need thereof, the regimen comprising administering one or more cancer therapies, wherein the regimen in an animal model achieves a stabilization or a reduction in the population of cancer stem cells. In a specific embodiment, the regimen achieves a 5%-40%, preferably a 10%-60%, and more preferably at 20 to 99% reduction in the cancer stem cell population in an immunodeficient mouse model, e.g., a severe combined immunodeficiency mouse model, as determined by a methods described infra. In some embodiments, the regimen achieves a 5%-40%, preferably a 10%-60%, and more preferably at 20 to 99% reduction in the cancer cell population. In some other embodiments, the regimen results in less than a 25%, preferably less than a 15%, and/or more preferably less than a 10% reduction in the circulating endothelial cell population and/or less than a 25%, preferably less than a 15%, and more preferably less than a 10% reduction in the circulating endothelial cell population and the circulating endothelial progenitor population. In a specific embodiment, the regimen achieves one or more such results after two weeks, a month, two months, three months, four months, six month, nine months, 1 year, 2 years, 3 years, 4 years, or more of administration of one or more of the therapies. In certain embodiments, the regimen comprises administering to the subject a dosage of one or more of the cancer therapies 1-5 times per day, twice a week, three times a week, four times a week, five times a week, weekly, twice a month, once a month or once every two to six months.

In another embodiment, the invention pertains to a therapy involving administration of a siRNA that is designed to halt expression of nanog.

The present invention also provides methods for treating cancer, the methods comprising administering to a patient (e.g., a human patient) in need thereof, a therapeutically effective regimen, the regimen comprising administering to the patient an antibody of the invention and one or more additional therapies, said additional therapy not being compounds of the invention. The compound of the invention and the additional therapy can be administered separately, concurrently, or sequentially. The combination of agents can act additively or synergistically.

Any therapy which is useful, has been used, or is currently being used for the treatment of cancer can be used in compositions and method of the invention. Therapies include, but are not limited to, peptides, antibodies, polypeptides, fusion proteins, nucleic acid molecules, small molecules, mimetic agents, synthetic drugs, inorganic molecules, vaccines, antibodies and organic molecules. Non-limiting examples of cancer therapies include chemotherapies, radiation therapies, hormonal therapies, small molecule therapies, toxin therapies, demethylation therapies, histone deacetylase inhibitor therapies, targeted therapies, epigenetic therapies, differentiation therapies, antiangiogenic therapies, biologic therapies, immunotherapies, or surgery. In certain embodiments, a therapeutically effective regimen of the invention comprises the administration of a combination of therapies.

Any therapy which is acting on a target or is an antibody belonging to one of the classes named below in this paragraph may be used in compositions and methods of the invention. Non limiting examples of agents, such as those that target or affect cancer stem cells, include: inhibitors of interleukin-3 receptor (IL-3R) and CD123 (including peptides, peptide-conjugates, antibodies, antibody-conjugates, antibody fragments, and antibody fragment-conjugates that target IL-3R or CD123), cantharidin, norcantharidin and analogs and derivatives thereof, Notch pathway inhibitors including gamma secretase inhibitors, sonic hedgehog/smoothened pathway inhibitors including cyclopamine and analogs thereof, antibodies to CD96, certain NF-kB/proteasome inhibitors including parthenolide and analogs thereof, certain triterpenes including celastrol, certain mTOR inhibitors, compounds and antibodies that target the urokinase receptor, sinefungin, certain inosine monophosphate dehydrogenase (IMPDH) inhibitors, PPAR-alpha and PPAR-gamma agonists and antagonists (including pioglitazone, tesaslitazar, muraglitazar, peliglitazar, lobeglitazone, balaglitazone, ragaglitazar, rosiglitazone, farglitazar, sodelglitazar, reglitazar, naveglitazar, oxeglitazar, metaglidasen, netoglitazone, darglitazone, englitazone, thiazolidinediones, aleglitazar, edaglitazone, rivoglitazone, troglitazone, imiglitazar, and sipoglitazar) telomerase inhibitors, antibodies to EpCAM (ESA), GSK-3 beta agonists and antagonists (including Lithium, 6-bromoinirubin-3′-oxime (BIO), TDZD8), Wnt pathway inhibitors including antibodies to frizzled or small molecules that inhibit disheveled/frizzled or beta catenin, anti-CD20 antibodies and conjugates (e.g. Rituxan, Bexxar, Zevalin) for novel use in multiple myeloma or melanoma, anti-CD133 antibody, anti-CD44 antibody, antibodies to IL-4, certain differentiation agents such as versnarinone, compounds that target CD33 such as an antibody or betulinic acid, compounds that target lactadherin such as an antibody, small molecules or antibodies that target CXCR4 or SDF-1, small molecules or antibodies that target multi-drug resistance pumps, inhibitors of survivin, inhibitors of XIAP, small molecules that target Bc1-2, antibodies to CLL-1, furin inhibitors (such as cucurbitacins).

An additional non-limiting list of compounds that could also be used to target cancer stem cells includes i) antibodies, antibody fragments, and proteins that are either naked or conjugated to a therapeutic moiety that target certain cell surface targets on cancer stem cells, or ii) small molecules known in the art including ones that can be further optimized (e.g., via chemistry) or identified via a cancer stem cell-based screen (e.g. such as one that would determine whether an antibody impairs proliferation or viability of a cancer stem cell through standard methods, the cell surface and intracellular targets including (not meant to be exhaustive) are: Rex1 (Zfp42), CTGF, Activin A, Wnt, FGF-2, HIF-1, AP-2gamma, Bmi-1, nucleostemin, hiwi, Moz-TIF2, Nanog, beta-arrestin-2, Oct-4, Sox2, stella, GDF3, RUNX3, EBAF, TDGF-1, nodal, ZFPY, PTNE, Evi-1, Pax3, Mc1-1, c-kit, Lex-1, Zfx, lactadherin, aldehyde dehydrogenase, BCRP, telomerase, CD133, Bc1-2, CD26, Gremlin, and FoxC2.

Examples of cancer therapies include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthracycline; anthramycin; asparaginase; asperlin; azacitidine (Vidaza); azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandronate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine (Ara-C); dacarbazine; dactinomycin; daunorubicin hydrochloride; decitabine (Dacogen); demethylation agents; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; EphA2 inhibitors; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; herceptin; histone deacetylase inhibitors (HDACs); hydroxyurea; idarubicin hydrochloride; ifosfamide; ilmofosine; imatinib mesylate (Gleevec, Glivec); interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; lenalidomide (Revlimid); letrozole; leuprolide acetate; liarozole hydrochloride; lometrexol sodium; lomustine; losoxantrone hydrochloride; masoprocol; maytansine; mechlorethamine hydrochloride; anti-CD2 antibodies (e.g., siplizumab (MedImmune Inc.; International Publication No. WO 02/098370, which is incorporated herein by reference in its entirety)); megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxaliplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other examples of cancer therapies include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP (Biogen, Cambridge, Mass.; International Publication No. WO 93/0686 and U.S. Pat. No. 6,162,432); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maytansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+mycobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; ocreotide; okicenone; oligonucleotides; onapristone; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene therapeutically effective regimens; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RH retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; gamma secretase inhibitors, single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; anti-integrin antibodies (e.g., anti-integrin .alpha..sub.v.beta..sub.3 antibodies); vorozole; zanoterone; zeniplatin; zilascorb; and zinostatin stimalamer.

In some embodiments, the therapy(ies) used in combination with an antibody of the invention is an immunomodulatory agent. Non-limiting examples of immunomodulatory agents include proteinaceous agents such as cytokines, peptide mimetics, and antibodies (e.g., human, humanized, chimeric, monoclonal, polyclonal, Fvs, ScFvs, Fab or F(ab).sub.2 fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, cytokine receptor modulators, and modulators mast cell modulators. Other examples of immunomodulatory agents can be found, e.g., in U.S. Publication No. 2005/0002934 A1 at paragraphs 259-275 which is incorporated herein by reference in its entirety. In one embodiment, the immunomodulatory agent is a chemotherapeutic agent. In an alternative embodiment, the immunomodulatory agent is an immunomodulatory agent other than a chemotherapeutic agent. In some embodiments, the therapy(ies) used in accordance with the invention is not an immunomodulatory agent.

### Polynucleotides and Expression Products

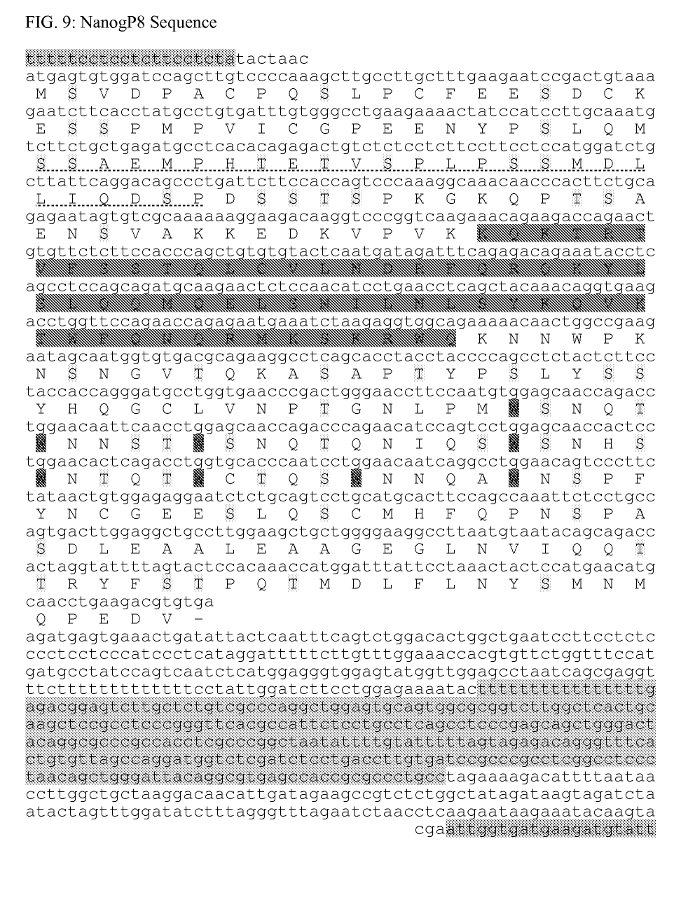

In the context of the present application, a polynucleotide sequence is “homologous” with the sequence according to the invention if at least 70%, preferably at least 80%, most preferably at least 90% of its base composition and base sequence corresponds to the sequence according to the invention. According to the invention, a “homologous protein” is to be understood to comprise proteins which contain an amino acid sequence at least 70% of which, preferably at least 80% of which, most preferably at least 90% of which, corresponds to the amino acid sequence shown in FIG. 9; wherein corresponds is to be understood to mean that the corresponding amino acids are either identical or are mutually homologous amino acids. The expression “homologous amino acids” denotes those which have corresponding properties, particularly with regard to their charge, hydrophobic character, steric properties, etc. Thus, in one embodiment the protein may be from 70% up to less than 100% homologous to nanog.

Homology, sequence similarity or sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

The term “isolated” means separated from its natural environment.

The term “polynucleotide” refers in general to polyribonucleotides and polydeoxyribonucleotides, and can denote an unmodified RNA or DNA or a modified RNA or DNA.

The term “polypeptides” is to be understood to mean peptides or proteins which contain two or more amino acids which are bound via peptide bonds.

The polypeptides for use in accord with the teachings herein include polypeptides corresponding to nanog, and also includes those, at least 70% of which, preferably at least 80% of which, are homologous with the polypeptide corresponding to nanog, and most preferably those which exhibit a homology of least 90% to 95% with the polypeptide corresponding to nanog and which have dedifferentiating influence. See polypeptide sequence provided in FIG. 9. Thus, the polypeptides may have a homology of from 70% to up to 100% with respect to nanog.

As used herein, a “polypeptide sequence exhibiting dedifferentiating influence” is a polypeptide whose presence in the cell causes an increase in potency, or transformation from a less developmentally potent cell to a more developmentally potent cell. Examples of such polypeptide sequences include the expression products of the nanog gene, and polynucleotide sequences that hybridize to the complement of the sequence in FIG. 9, as well as expression products of the polynucleotide sequences listed in Table 1 below in Example 3.

The terms “stringent conditions” or “stringent hybridization conditions” includes reference to conditions under which a polynucleotide will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaC1, 1% SDS (sodium dodecyl sulphate) at 37° C., and a wash in lx to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tm can be approximated from the equation of Meinkoth and Wahl, Anal. Biochem., 138:267-284 (1984): Tm=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with approximately 90% identity are sought, the Tm can be decreased 10° C. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution) it is preferred to increase the SSC concentration so that a higher temperature can be used. An extensive guide to the hybridization of nucleic acids is found in Current Protocols in Molecular Biology, Chapter 2, Ausubel, et al., Eds., Greene Publishing and Wiley-Interscience, New York (2000).

In a more specific embodiment, the activated dendritic cell lines are catalogued based on the cancer/tumor type used for activation along with at least one other characteristic, such as phenotypic information, morphological characteristics, differentiation profile, blood type, major histocompatibility complex, or genotypic information (e.g. single nucleated polymorphisms, ‘SNPs’ of a specific nucleic acid sequence associated with a gene, or genomic or mitochondrial DNA

The amount of cancer stem cells can be monitored/assessed using standard techniques known to one of skill in the art. Cancer stem cells can be monitored by, e.g., obtaining a sample, such as a tissue/tumor sample, blood sample or a bone marrow sample, from a subject and detecting cancer stem cells in the sample. The amount of cancer stem cells in a sample (which may be expressed as percentages of, e.g., overall cells or overall cancer cells) can be assessed by detecting the expression of antigens on cancer stem cells. Techniques known to those skilled in the art can be used for measuring these activities. Antigen expression can be assayed, for example, by immunoassays including, but not limited to, western blots, immunohistochemistry, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, immunofluorescence, protein A immunoassays, flow cytometry, and FACS analysis. In such circumstances, the amount of cancer stem cells in a test sample from a subject may be determined by comparing the results to the amount of stem cells in a reference sample (e.g., a sample from a subject who has no detectable cancer) or to a predetermined reference range, or to the patient him/herself at an earlier time point (e.g. prior to, or during therapy).

In a specific embodiment, the cancer stem cell population in a sample from a patient is determined by flow cytometry. This method exploits the differential expression of certain surface markers on cancer stem cells relative to the bulk of the tumor. Labeled antibodies (e.g., fluorescent antibodies) can be used to react with the cells in the sample, and the cells are subsequently sorted by FACS methods. In some embodiments, a combination of cell surface markers are utilized in order to determine the amount of cancer stem cells in the sample. For example, both positive and negative cell sorting may be used to assess the amount of cancer stem cells in the sample. Cancer stem cells for specific tumor types can be determined by assessing the expression of markers on cancer stem cells.

In certain embodiments using flow cytometry of a sample, the Hoechst dye protocol can be used to identify cancer stem cells in tumors. Briefly, two Hoechst dyes of different colors (typically red and blue) are incubated with tumor cells. The cancer stem cells, in comparison with bulk cancer cells, over-express dye efflux pumps on their surface that allow these cells to pump the dye back out of the cell. Bulk tumor cells largely have fewer of these pumps, and are therefore relatively positive for the dye, which can be detected by flow cytometry. Typically a gradient of dye positive (“dye.sup.+”) vs. dye negative (“dye.sup.−”) cells emerges when the entire population of cells is observed. Cancer stem cells are contained in the dye- or dye low (dye.sup.low) population. For an example of the use of the Hoechst dye protocol to characterize a stem cell or cancer stem cell population see Goodell, et al., Blood, 98(4):1166-1173 (2001) and Kondo, et al., Proc Natl Acad Sci USA 101:781-786 (2004). In this way, flow cytometry could be used to measure cancer stem cell amount pre- and post-therapy to assess the change in cancer stem cell amount arising from a given therapy or regimen.

In other embodiments using flow cytometry of a sample, the cells in the sample may be treated with a substrate for aldehyde dehydrogenase that becomes fluorescent when catalyzed by this enzyme. For instance, the sample can be treated with BODIPY™-aminoacetaldehyde which is commercially available from StemCell Technologies Inc. as Aldefluor™. Cancer stem cells express high levels of aldehyde dehydrogenase relative to bulk cancer cells and therefore become brightly fluorescent upon reaction with the substrate. The cancer stem cells, which become fluorescent in this type of experiment, can then be detected and counted using a standard flow cytometer. In this way, flow cytometry could be used to measure cancer stem cell amount pre- and post-therapy to assess the change in cancer stem cell amount arising from a given therapy or regimen.

In other embodiments, a sample (e.g., a tumor or normal tissue sample, blood sample or bone marrow sample) obtained from the patient is cultured in in vitro systems to assess the cancer stem cell population or amount of cancer stem cells. For example, tumor samples can be cultured on soft agar, and the amount of cancer stem cells can be correlated to the ability of the sample to generate colonies of cells that can be visually counted. Colony formation is considered a surrogate measure of stem cell content, and thus, can be used to quantitate the amount of cancer stem cells. For instance, with hematological cancers, colony-forming assays include colony forming cell (CFC) assays, long-term culture initiating cell (LTC-IC) assays, and suspension culture initiating cell (SC-IC) assays. In this way, the colony-forming or related assay could be used to measure cancer stem cell amount pre- and post-therapy to assess the change in cancer stem cell amount arising from a given therapy or regimen.

In other embodiments, sphere formation is measured to determine the amount of cancer stem cells in a sample (e.g., cancer stem cells form three-dimensional clusters of cells, called spheres) in appropriate media that is conducive to forming spheres. Spheres can be quantitated to provide a measure of cancer stem cells. See Singh, et al., Cancer Res 63: 5821-5828 (2003). Secondary spheres can also be measured. Secondary spheres are generated when the spheres that form from the patient sample are broken apart, and then allowed to reform. In this way, the sphere-forming assay could be used to measure cancer stem cell amount pre- and post-therapy to assess the change in cancer stem cell amount arising from a given therapy or regimen.

In other embodiments, the amount of cancer stem cells in a sample can be determined with a cobblestone assay. Cancer stem cells from certain hematological cancers form “cobblestone areas” (CAs) when added to a culture containing a monolayer of bone marrow stromal cells. For instance, the amount of cancer stem cells from a leukemia sample can be assessed by this technique. The tumor samples are added to the monolayer of bone marrow stromal cells. The leukemia cancer stem cells, more so than the bulk leukemia cells, have the ability to migrate under the stromal layer and seed the formation of a colony of cells which can be seen visually under phase contrast microscopy in approximately 10-14 days as CAs. The number of CAs in the culture is a reflection of the leukemia cancer stem cell content of the tumor sample, and is considered a surrogate measure of the amount of stem cells capable of engrafting the bone marrow of immunodeficient mice. This assay can also be modified so that the CAs can be quantitated using biochemical labels of proliferating cells instead of manual counting, in order to increase the throughput of the assay. See Chung, et al., Blood 105(1):77-84 (2005). In this way, the cobblestone assay could be used to measure cancer stem cell amount pre- and post-therapy to assess the change in cancer stem cell amount arising from a given therapy or regimen.

In other embodiments, a sample (e.g., a tumor or normal tissue sample, blood sample or bone marrow sample) obtained from the patient is analyzed in in vivo systems to determine the cancer stem cell population or amount of cancer stem cells. In certain embodiments, for example, in vivo engraftment is used to quantitate the amount of cancer stem cells in a sample. In vivo engraftment involves implantation of a human specimen with the readout being the formation of tumors in an animal such as in immunocompromised or immunodeficient mice (such as NOD/SCID mice). Typically, the patient sample is cultured or manipulated in vitro and then injected into the mice. In these assays, mice can be injected with a decreasing amount of cells from patient samples, and the frequency of tumor formation can be plotted vs. the amount of cells injected to determine the amount of cancer stem cells in the sample. Alternatively, the rate of growth of the resulting tumor can be measured, with larger or more rapidly advancing tumors indicating a higher cancer stem cell amount in the patient sample. In this way, an in vivo engraftment model/assay could be used to measure cancer stem cell amount pre- and post-therapy to assess the change in cancer stem cell amount arising from a given therapy or regimen.

In certain in vivo techniques, an imaging agent, or diagnostic moiety, is used which binds to molecules on cancer cells or cancer stem cells, e.g., cancer cell or cancer stem cell surface antigens. For instance, a fluorescent tag, radionuclide, heavy metal, or photon-emitter is attached to an antibody (including an antibody fragment) that binds to a cancer stem cell surface antigen. The medical practitioner can infuse the labeled antibody into the patient either prior to, during, or following treatment, and then the practitioner can place the patient into a total body scanner/developer which can detect the attached label (e.g., fluorescent tag, radionuclide, heavy metal, photon-emitter). The scanner/developer (e.g., CT, MRI, or other scanner, e.g. detector of fluorescent label, that can detect the label) records the presence, amount/quantity, and bodily location of the bound antibody. In this manner, the mapping and quantitation of tag (e.g. fluorescence, radioactivity, etc.) in patterns (i.e., different from patterns of normal stem cells within a tissue) within a tissue or tissues indicates the treatment efficacy within the patient's body when compared to a reference control such as the same patient at an earlier time point or a patient or healthy individual who has no detectable cancer. For example, a large signal (relative to a reference range or a prior treatment date, or prior to treatment) at a particular location indicates the presence of cancer stem cells. If this signal is increased relative to a prior date it suggests a worsening of the disease and failure of therapy or regimen. Alternatively, a signal decrease indicates that the therapy or regimen has been effective.

In a specific embodiment, the amount of cancer stem cells is detected in vivo in a subject according to a method comprising the steps of: (a) administering to the subject an effective amount of a labeled cancer stem cell marker binding agent that binds to a cell surface marker found on the cancer stem cells, and (b) detecting the labeled agent in the subject following a time interval sufficient to allow the labeled agent to concentrate at sites in the subject where the cancer stem cell surface marker is expressed. In accordance with this embodiment, the cancer stem cell surface marker-binding agent is administered to the subject according to any suitable method in the art, for example, parenterally (such as intravenously), or intraperitoneally. In another embodiment, the cancer stem cell surface marker-binding agent is administered to the subject according to any suitable method in the art, for example, locally (such as directly into the lumen of the bladder), intratumorally or intraperitoneally. In accordance with this embodiment, the effective amount of the agent is the amount which permits the detection of the agent in the subject. This amount will vary according to the particular subject, the label used, and the detection method employed. For example, it is understood in the art that the size of the subject and the imaging system used will determine the amount of labeled agent needed to detect the agent in a subject using an imaging means. In the case of a radiolabeled agent for a human subject, the amount of labeled agent administered is measured in terms of radioactivity, for example from about 5 to 20 millicuries of .sup.99Tc. The time interval following the administration of the labeled agent which is sufficient to allow the labeled agent to concentrate at sites in the subject where the cancer stem cell surface marker is expressed will vary depending on several factors, for example, the type of label used, the mode of administration, and the part of the subject's body that is imaged. In a particular embodiment, the time interval that is sufficient is 6 to 48 hours, 6 to 24 hours, or 6 to 12 hours. In another embodiment the time interval is 5 to 20 days or 5 to 10 days. The presence of the labeled cancer stem cell surface marker-binding agent can be detected in the subject using imaging means known in the art. In general, the imaging means employed depend upon the type of label used. Skilled artisans will be able to determine the appropriate means for detecting a particular label. Methods and devices that may be used include, but are not limited to, computed tomography (CT), whole body scan such as position emission tomography (PET), magnetic resonance imaging (MRI), and sonography. In a specific embodiment, the cancer stem cell surface marker-binding agent is labeled with a radioisotope and is detected in the patient using a radiation responsive surgical instrument (Thurston, et al., U.S. Pat. No. 5,441,050). In another embodiment, the cancer stem cell surface marker-binding agent is labeled with a fluorescent compound and is detected in the patient using a fluorescence responsive scanning instrument. In another embodiment, the cancer stem cell surface marker-binding agent is labeled with a positron emitting metal and is detected in the patient using positron emission-tomography. In yet another embodiment, the cancer stem cell surface marker-binding agent is labeled with a paramagnetic label and is detected in a patient using magnetic resonance imaging (MRI).

Any in vitro or in vivo (ex vivo) assays known to those skilled in the art that can detect and/or quantify cancer stem cells can be used to monitor cancer stem cells in order to evaluate the prophylactic and/or therapeutic utility of a cancer therapy or regimen disclosed herein for cancer or one or more symptoms thereof; or these assays can be used to assess the prognosis of a patient. The results of these assays then may be used to possibly maintain or alter the cancer therapy or regimen.

The amount of cancer stem cells in a specimen can be compared to a predetermined reference range and/or an earlier amount of cancer stem cells previously determined for the subject (either prior to, or during therapy) in order to gauge the subject's response to the treatment regimens described herein. In a specific embodiment, a stabilization or reduction in the amount of cancer stem cells relative to a predetermined reference range and/or earlier cancer stem cell amount previously determined for the subject (prior to, during and/or after therapy) indicates that the therapy or regimen was effective and thus possibly an improvement in the subject's prognosis, whereas an increase relative to the predetermined reference range and/or cancer stem cell amount detected at an earlier time point indicates that the therapy or regimen was ineffective and thus possibly the same or a worsening in the subject's prognosis. The cancer stem cell amount can be used with other standard measures of cancer to assess the prognosis of the subject and/or efficacy of the therapy or regimen: such as response rate, durability of response, relapse-free survival, disease-free survival, progression-free survival, and overall survival. In certain embodiments, the dosage, frequency and/or duration of administration of a therapy is modified as a result of the determination of the amount or change in relative amount of cancer stem cells at various time points which may include prior to, during, and/or following therapy.

The present invention also relates to methods for determining that a cancer therapy or regimen is effective at targeting and/or impairing cancer stem cells by virtue of monitoring cancer stem cells over time and detecting a stabilization or decrease in the amount of cancer stem cells during and/or following the course of the cancer therapy or regimen.

In a certain embodiment, a therapy or regimen may be marketed as an anti-cancer stem cell therapy or regimen based on the determination that a therapy or regimen is effective at targeting and/or impairing cancer stem cells by virtue of having monitored or detected a stabilization or decrease in the amount of cancer stem cells during therapy.

U.S. Patent Publications 20070071731; 20060188489; 20060099193; and 20060134789 20080102521 are cited for further discussion of stem cells, and experimental protocols related thereto. US Patent Pub 20080118518 is cited for use of isolated cancer stem cells and using the knowledge that nanog is differentially expressed therein for screening new potential drug candidates. 20090081214 is cited for further discussion of using a marker, such as the newly discovered nanog, to develop novel cancer therapies. The sequence submitted with the instant application includes the genetic and protein sequence of nanog.

In reviewing the detailed disclosure which follows, and the specification more generally, it should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skilled in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

1. A method for treating or preventing the reoccurence of cancer in a subject, said method comprising administering a therapeutically effective amount of a nanog modulating agent to said subject.

2. The method of claim 1, wherein said nanog modulating agent is an antibody that binds to nanog.

3. The method of claim 1, wherein said nanog modulating agent is a nanog antibody conjugate.

4. The method of claim 1, wherein said nanog modulating agent is an siRNA that binds to a polynucleotide that encodes nanog.

5. The method of claim 1, further comprising administering an additional therapy to said subject prior to, during or subsequent to said administering of said nanog modulating agent.

6. A method of diagnosing a disease characterized by expression or abnormal expression of a tumor-associated antigen, which method comprises(i) detection of a nucleic acid which codes for the tumor-associated antigen or of a part thereof, and/or(ii) detection of the tumor-associated antigen or of a part thereof, and/or(iii) detection of an antibody to the tumor-associated antigen or of a part thereof and/or(iv) detection of cytotoxic or T helper lymphocytes which are specific to the tumor-associated antigen or to a part thereof in a biological sample isolated from a patient, with said tumor-associated antigen said tumor antigen being nanog or a polypeptide molecule having at least 80, 85, 90 or 95 percent identity thereto, encoded by a polynucleotide having a sequence encoded by a nucleic acid sequence relating to the human nanog gene.

7. A method of screening for therapeutic agents useful in the treatment of cancer in a mammal comprising the steps of i) contacting a test compound with a cancer stem cell expressing a nanog polypeptide and ii) detecting a deleterious effect on said cancer stem cell, wherein a test compound which shows a deleterious effect is identified as a potential therapeutic agent for killing, differentiating or weakening a nanog expressing cancer stem cell.

8. The method of claim 7, wherein said therapeutic agent causes said nanog expressing cancer stem cell to cease expressing nanog.

9. The method of claim 8, wherein ceasing expression of nanog causes said cancer stem cell to become a more rapidly dividing cell.

10. A pharmaceutical composition for the treatment of cancer in a mammal comprising a nanog modulating agent, wherein said nanog modulating agent is i) a small molecule, ii) an RNA molecule, iii) an antisense oligonucleotide, iv) a polypeptide, v) an antibody, or vi) a ribozyme.

11. The composition of claim 10, further comprising a pharmaceutically acceptable carrier.

12. Method for the preparation of a pharmaceutical composition useful for the treatment of cancer in a mammal comprising the steps of i) identifying a therapeutic agent in accord with the method of claim 7; ii) determining whether said therapeutic ameliorates the cancer in a mammal; and iii) combining of said therapeutic agent with an acceptable pharmaceutical carrier.

13. The method of claim 1, wherein the cancer is breast cancer, testicular cancer, lung cancer, melanoma, brain cancer, myeloma, Hodgkin's disease, hepatoma, stomach cancer, bladder cancer, uterine cancer, neuroblastoma, thyroid cancer, sarcoma, cervical cancer, Wilm's tumor, colorectal cancer, pancreatic cancer, skin cancer, prostate cancer, ovarian cancer, kidney cancer, lymphoma, acute myelogenous leukemia, acute lymphocytic leukemia, multiple myeloma, ependymoma, chronic lymphocytic leukemia, myelodysplastic syndrome, or chronic myelogenous leukemia.

14. A method for preventing, treating, or managing cancer resulting in a reduction in bulk tumor size and/or a reduction in cancer cells, the method comprising identifying the presence of cancer stem cells expressing nanog in a tumor in a human subject, administering to said human subject in need thereof a prophylactically or therapeutically effective regimen, the regimen comprising the administration of a therapeutic composition according to claim 10 to the human subject, and monitoring changes in the amount of said cancer stem cells, wherein the regimen results in at least an approximately 10% reduction in cancer stem cells in said human subject.
